(19) United States
(12) Patent Application Publication
(10) Pub. No.: US 2014/0273121 A1
(43) Pub. Date: Sep. 18, 2014

(54) Method for production of n-propanol and other C3-containing products from syngas using membrane supported bioreactor

(71) Applicants: Rathin Datta, Chicago, IL (US); Michael Enzien, Lisle, IL (US); Jian Xu, Naperville, IL (US); Young Do, Naperville, IL (US); William Levinson, Naperville, IL (US)

(72) Inventors: Rathin Datta, Chicago, IL (US); Michael Enzien, Lisle, IL (US); Jian Xu, Naperville, IL (US); Young Do, Naperville, IL (US); William Levinson, Naperville, IL (US)

(21) Appl. No.: 13/802,924

(22) Filed: Mar. 14, 2013

(51) Int. Cl. C12P 7/04; C12P 7/52

(52) U.S. Cl. CPC C12P 7/04; C12P 7/52
USPC 435/141; 435/157

(57) ABSTRACT

This invention provides methods for the production of n-propanol. Specifically, the methods and systems of the present invention use symbiotic co-cultures operating in a membrane supported bioreactor for the production of n-propanol from syngas.

### FIELD OF THE INVENTION

The invention provides methods for production of n-propanol and other C3-containing products from syngas using symbiotic co-cultures of anaerobic microorganisms operating in a membrane supported bioreactor.

### BACKGROUND OF THE INVENTION

Propanol is a solvent used industrially, but more importantly, it can be readily dehydrated to produce propylene which is the second largest chemical commodity in the world with production of >70 million tons/per year. Currently propylene is produced mainly by steam-cracking of naphtha or liquid petroleum gas or fluid catalytic cracking of gasoils in very large installations as a secondary product. The steam-cracking is a process that makes mainly ethylene and many other co-products, such as butylenes, butadiene and pyrolysis gasoline all of which need to be purified and to be utilized simultaneously. Other ways to make propylene is in a refinery FCC (fluid catalytic cracking) where propylene is a byproduct from heavy gasoil cracking in proportions between 3 and 15 wt %. Propylene can also be produced by catalytic dehydrogenation of propane. Still another way to make propylene is via metathesis of butenes with ethylene.

Since for many centuries, simple sugars are being fermented into ethanol with the help of saccharomyces cerevisae. The last decade's new routes starting from cellulose and hemicelluloses have been developed to ferment more complex carbohydrates into ethanol. Hereto, the carbohydrates need to be unlocked from the lignocellulosic biomass. Biomass consists approximately of 30% cellulose, 35% hemicelluloses and 25% lignin. The lignin fraction cannot be valorised as ethanol because of its aromatic nature, and can only be used as an energy source which is present in many cases in excess for running an industrial plant.

Several microorganisms are able to use one-carbon compounds as a carbon source and some even as an energy source. Carbon dioxide is an important carbon source for phototrophs, sulfate reducers, methanogens, acetogens and chemolithotrophic microorganisms. There are essentially four systems to fix CO₂: (1) the Calvin cycle [CO₂ fixing enzyme: ribulose-1,5-bisphosphate carboxylase], (2) the reductive citric acid cycle [CO₂ fixing enzymes: 2-oxoglutarate synthase, isocitrate dehydrogenase, pyruvate synthase], (3) the acetyl-CoA pathway [CO₂ fixing enzyme: acetyl-CoA synthase, linked to CO-dehydrogenase] and (4) the 3-hydroxypropionate cycle [CO₂ fixing enzyme: acetyl-CoA carboxylase, propionyl-CoA carboxylase] (“Structural and functional relationships in Prokaryotes”, L. Barton, Springer 2005; “Carbon monoxide-dependent energy metabolism in anaerobic bacteria and archaea”, E. Oelgeschelager, M. Rother, Arch. Microbiol., 190, p. 257, 2008; “Life with carbon monoxide”, S. Ragsdale, Critical Reviews in Biochem. and Mol. Biology, 39, p. 165, 2004).

More recently, more efficient routes that produce synthesis gas from carbon-containing materials and that subsequently is fermented into ethanol are being developed (“B10 conversion of synthesis gas into liquid or gaseous fuels”, K. Klasson, M. Ackerson, E. Clausen, J. Gaddy, Enzyme and Microbial Technology, 14(8), p. 602, 1992; “Fermentation of Biomass-Generated Producer Gas to Ethanol”, R. Datar, R. Shenkman, B. Cateni, R. Huhnke, R. Lewis, Biotechnology and Bioengineering, 86 (5), p. 587, 2004; “Microbiology of synthesis gas fermentation for biofuel production”, A. Hemstra, J. Sipma, A. Rinzema, A. Stams, Current Opinion in Biotechnology, 18, p. 200, 2007; “Old Acetogens, New Light”, H. Drake, A. Goöβner, S. Daniel, Ann. N.Y. Acad. Sci. 1125: 100-128, 2008). Synthesis gas can be produced by gasification of the whole biomass without need to unlock certain fractions. Synthesis gas can also be produced from other feedstock via gasification: (i) coal, (ii) municipal waste (iii) plastic waste, (iv) petcoke and (v) liquid residues from refineries or from the paper industry (black liquor). Synthesis gas can also be produced from natural gas via steamreforming or autothermal reforming (partial oxidation).

The biochemical pathway of synthesis gas conversion is described by the Wood-Ljungdahl Pathway. Fermentation of syngas offers several advantages such as high specificity of biocatalysts, lower energy costs (because of low pressure and low temperature bioconversion conditions), greater resistance to biocatalyst poisoning and nearly no constraint for a preset H₂ to CO ratio (“Reactor design issues for synthesis-gas fermentations” M. Bredwell, P. Srivastava, R. Worden, Biotechnology Progress 15, 834-844, 1999; “Biological conversion of synthesis gas into fuels”, K. Klasson, C. Ackerson, E. Clausen, J. Gaddy, International Journal of Hydrogen Energy 17, p. 281, 1992). Acetogens are a group of anaerobic bacteria able to convert syngas components, like CO, CO₂ and H₂ to acetate via the reductive acetyl-CoA or the Wood-Ljungdahl pathway.

Several anaerobic bacteria have been isolated that have the ability to ferment syngas to ethanol, acetic acid and other useful end products. Clostridium ljungdahlii and Clostridium autoethanogenum, were two of the first known organisms to convert CO, CO₂ and H₂ to ethanol and acetic acid. Commonly known as acetogens, these microorganisms have the ability to reduce CO₂ to acetate in order to produce required energy and to produce cell mass. The overall stoichiometry for the synthesis of ethanol using three different combinations of syngas components is as follows (J. Vega, S. Prieto, B. Elmore, E. Clausen, J. Gaddy, “The Biological Production of Ethanol from Synthesis Gas,” Applied Biochemistry and Biotechnology, 20-1, p. 781, 1989):

6CO+3H₂O→CH₃CH₂OH+4CO₂

2CO₂+6H₂→CH₃CH₂OH+3H₂O

6CO+6H₂→2CH₃CH₂OH+2CO₂

The primary product produced by the fermentation of CO and/or H₂ and CO₂ by homoacetogens is ethanol principally according to the first two of the previously given reactions. Homoacetogens may also produce acetate. Acetate production occurs via the following reactions:

4CO+2H₂O→CH₃COOH+2CO₂

4H₂+2CO₂→CH₃COOH+2H₂O

Clostridium ljungdahlii, one of the first autotrophic microorganisms known to ferment synthesis gas to ethanol was isolated in 1987, as an homoacetogen it favors the production of acetate during its active growth phase (acetogenesis) while ethanol is produced primarily as a non-growth-related product (solventogenesis) (“Biological conversion of synthesis gas into fuels”, K. Klasson, C. Ackerson, E. Clausen, J. Gaddy, International Journal of Hydrogen Energy 17, p. 281, 1992).

Clostridium autoethanogenum is a strictly anaerobic, gram-positive, spore-forming, rod-like, motile bacterium which metabolizes CO to form ethanol, acetate and CO₂ as end products, beside it ability to use CO₂ and H₂, pyruvate, xylose, arabinose, fructose, rhamnose and L-glutamate as substrates (J. Abrini, H. Naveau, E. Nyns, “Clostridium autoethanogenum, Sp-Nov, an Anaerobic Bacterium That Produces Ethanol from Carbon-Monoxide”, Archives of Microbiology, 161(4), p. 345, 1994).

Anaerobic acetogenic microorganisms offer a viable route to convert waste gases, such as syngas, to useful products, such as ethanol, via a fermentation process. Such bacteria catalyze the conversion of H₂ and CO₂ and/or CO to acids and/or alcohols with higher specificity, higher yields and lower energy costs than can be attained by traditional production processes. While many of the anaerobic microorganisms utilized in the fermentation of ethanol also produce a small amount of propanol as a by-product, to date, no single anaerobic microorganism has been described that can utilize the fermentation process to produce high yields of propanol.

Therefore a need in the art remains for methods using microorganisms in the production of propanol using fermentation with a substrate of H₂ and CO₂ and/or CO.

### SUMMARY OF THE INVENTION

A method is disclosed for producing propanol and/or propionate by exposing gaseous substrates selected from the group consisting of carbon monoxide, carbon dioxide and hydrogen or combinations thereof to a symbiotic co-culture comprising a C1-fixing microorganism and a C3-producing microorganism both contained within biopores that open to a gas contact surface located on one side of an asymmetric hydrophilic membrane. The method circulates a fermentation liquid on a liquid contact surface located on the side of the membrane opposite to the gas contact surface to supply nutrients to the biopores and to recover propanol from the biopores while maintaining the symbiotic co-culture at conditions effective to convert the gaseous substrate into propanol.

The C1-fixing and the C3 producing microorganisms may be introduced into the biopores in a variety of ways. The method may introduce a mixture of the C1-fixing microorganism and the C3-producing microorganism at the same time into the biopores of the asymmetric membrane. Or, the method may introduce the C1-fixing microorganism and the C3-producing microorganism by sequential seeding into the biopores that first one of the microorganisms and then another of the microorganisms into the biopores. For example, the method may first introduce a culture of the C3-producing microorganism into the biopores and subsequently introduce a culture of the C1-fixing microorganism into the biopores or the order of the seeding may be reversed to first introduce the C1 fixing microorganism and then the C3 producing microorganism. In most cases the C3-producing microorganism is a propionogen and the gaseous substrate is syngas.

### BRIEF DESCRIPTION OF THE DRAWINGS

These and other objects, features, and embodiments of the invention will be better understood from the following detailed description taken in conjunction with the drawings, wherein:

FIG. 1 is a schematic diagram of an embodiment of the symbiotic association of anaerobic microorganism cultures of the invention. The C1-fixing microorganism produces ethanol and acetate from syngas. The symbiotic C3-producing microorganism coverts the ethanol, acetate and (secondarily H₂/CO/CO₂) to C3-containing products, namely propionate and propanol. The C1-fixing microorganism also converts the propionate to propanol, which becomes the primary end product.

FIG. 2 is a detailed illustration of the methylmalonyl-succinate pathway used by anaerobic microorganisms for C3 (propionate) production.

FIG. 3 is a detailed illustration of the lactate-acrylate pathway used by anaerobic microorganisms for C3 (propionate/propanol) production.

FIG. 4 illustrates the symbiotic MSBR. The propionogen organisms are mixed with the homoacetogen organisms and one type of microorganism is depicted by the open circles and another type of microorganism is depicted by the black circles. The schematic diagram shows sequential seeding of microorganisms on the right side of the Figure and simultaneous seeding of a mixture of the microorganisms on the left side of the Figure. The Figure also shows the spatial relationship between the C1 and C3 microorganisms, and the gas and liquid flow for a single hollow fiber in an MSBR membrane.

FIG. 5 illustrates a specific configuration of a bio-reactor system utilizing the membrane containing microorganisms as depicted in FIG. 4.

### DETAILED DESCRIPTION OF THE INVENTION

The invention provides methods for the production of propanol and other C3-containing products from syngas by symbiotic co-cultures of anaerobic microorganisms operating in a membrane supported bioreactor (MSBR).

As used herein, synthesis gas (syngas) is a gas containing carbon monoxide, carbon dioxide and frequently hydrogen . “Syngas” includes streams that contain carbon dioxide in combination with hydrogen and that may include little or no carbon monoxide. “Syngas” may also include carbon monoxide gas streams that may have little or no hydrogen.

As used herein, the term “symbiotic” refers to the association of two or more different types (e.g. organisms, populations, strains, species, genera, families, etc.) of anaerobic microorganisms which are capable of forming a tightly associated metabolic symbiosis. As used herein, the term “co-culture” of microorganisms refers to joint incubation or incubation together, of the symbiotic microorganisms. In the context of the present invention, the co-culture does not require cellular population growth during the joint incubation of the symbiotic microorganisms.

In an embodiment of the invention illustrated in FIG. 1, two types of anaerobic microorganism are utilized to create the symbiotic co-culture for production of propanol. The first type of microorganism in the symbiotic co-culture is a primary C1-fixing microorganism, which utilizes syngas as the sole carbon and electron source and produces ethanol and acetate as the dissimilatory metabolite products. The second type of microorganism in the symbiotic co-culture is capable of growing on the dissimilatory metabolites of the C1-fixing microorganism (ethanol and acetate) as its sole carbon and/or electron source to produce a C3-carbon molecule, such as propanol or propionic acid, as its primary product or together with syngas (as additional carbon and/or electron source) convert the metabolites of the C1-carbon fixing microorganism to C3-carbon molecules. This second microorganism shall be referred to herein as the C3-producing microorganism. Advantageously, the C1-fixing microorganism may also be capable of converting the propionate produced by the C3-producing microorganism into propanol.

The C1-fixing microorganisms of the invention are also homoacetogens. Homoacetogens have the ability, under anaerobic conditions, to produce acetic acid and ethanol from the substrates, CO+H₂O, or H₂+CO₂ or CO+H₂+CO₂. The CO or CO₂ provide the carbon source and the H₂ or CO provide the electron source for the reactions producing acetic acid and ethanol. The primary product produced by the fermentation of CO and/or H₂ and CO₂ by homoacetogens is principally ethanol according to the two previously given reactions so that the C1 fixing microorganisms are acting as solventogenic homoacetogens using the acetyl-CoA pathway.

6CO+3H₂O→C₂H₅OH+4CO₂

6H₂+2CO₂C₂H₅OH+3H₂O

Homoacetogens may also produce acetate. Acetate production occurs via the following reactions:

4CO+2H₂O→CH₃COOH+2CO₂

4H₂+2CO₂→CH₃COOH+2H₂O

C1-fixing microorganisms suitable for use in the inventive method include, without limitation, homoacetogens such as Clostridium ljungdahlii, Clostridium autoethanogenum, Clostridium ragsdalei, and Clostridium coskatii. Additional C1 fixing microorganisms that are suitable for the invention include Alkalibaculum bacchi, Clostridium thermoaceticum, and Clostridium aceticum.

In this invention the symbiotic C3-producing microorganisms are capable of growing on ethanol and/or acetate as their primary carbon source. These microorganisms include, but are not limited to the organisms and their pathways described above and include: Pelobacter propionicus, Clostridium neopropionicum, Clostridium propionicum, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosymbioticum, Pelotomaculum thermopropionicum, and Pelotomaculum schinkii. In particular embodiments of the invention, the C3-producing microorganisms are propionogens. Propionogens refers to any microorganism capable of converting syngas intermediates, such as ethanol and acetate, to propionic acid and propanol. Propionogens of the invention utilize one of at least two distinct pathways for the conversion of acetate and ethanol to propionate—the methylmalonyl-succinate pathway (shown in FIG. 2) and the lactate-acrylate pathway (shown in FIG. 3).

Pelobacter propionicus, using the dicarboxylic acid pathway, has been shown to grow on ethanol as substrate while producing propionate in presence of CO2 (Schink, B., Kremer, D. and Hansen, T., “Pathway of propionate formation from ethanol in Pelobacter propionicus”, Arch. Microbiol. 147, 321-327, 1987 and S. Seeliger, P. Janssen, B. Schink, “Energetics and kinetics of lactate fermentation to acetate and propionate via methylmalonyl-CoA or acrylyl-CoA”, FEMS Microbiology Letters, 211, pp. 65-70, 2002). When ethanol is fed together with CO2 and hydrogen, significant amounts of propanol are produced. Ethanol is converted into acetyl-CoA (via acetaldehyde) while producing electrons for the carboxylation of acetyl-CoA into pyruvate, catalysed by pyruvate synthase. Combined with the dicarboxylic acid pathway propionate is produced from ethanol and CO2 (Schink et al., 1987).

3ethanol+2HCO₃-→2propionate-+acetate-+H++3H₂0

Pelobacter propionicus is not able to reductively convert acetate and CO2 into propionate whereas Desulfobulbus propionicus does make propionate from acetate and CO2 (Schink et al., 1987).

acetate-+HCO₃-+3H₂→propionate-+3H₂0

Clostridium neopropionicum (strain X4), using the acrylate pathway, is able to convert ethanol and CO₂ into acetate, propionate and some propanol (J. Tholozan, J. Touzel, E. Samain, J. Grivet, G. Prensier and G. Albagnac, “Clostridium neopropionicum sp. Nov., a strict anaerobic bacterium fermenting ethanol to propionate through acrylate pathway”, Arch. Microbiol., 157, p. 249-257, 1992). As for the dicarboxylic acid pathway, the intermediate acetyl-CoA produced from the substrate ethanol is linked to the acrylate pathway via the pyruvate synthase that converts acetyl-CoA into pyruvate by carboxylation with CO₂.

The symbiotic cultures of the present invention have the capability as co-cultures to produce propanol from gaseous carbon and electron sources. Suitable sources of carbon and electron sources for the cultures include “waste” gases such as syngas, oil refinery waste gases, steel manufacturing waste gases, gases produced by steam, autothermal or combined reforming of natural gas or naphtha, biogas and products of biomass, coal or refinery residues gasification or mixtures of the latter. Sources also include gases (containing some H₂) which are produced by yeast, clostridial fermentations, and gasified cellulosic materials. Such gaseous substrates may be produced as byproducts of other processes or may be produced specifically for use in the methods of the present invention. Those of skill in the art will recognize that any source of substrate gas may be used in the practice of the present invention, so long as it is possible to provide the microorganisms of the co-culture with sufficient quantities of the substrate gases under conditions suitable for the bacterium to carry out the fermentation reactions.

In one preferred embodiment of the invention, the source of CO, CO₂ and H₂ is syngas. Syngas for use as a substrate may be obtained, for example, as a gaseous product of coal or refinery residues gasification. Syngas may also be produced by reforming natural gas or naphtha, for example by the reforming of natural gas in a steam methane reformer. Alternatively, syngas can be produced by gasification of readily available low-cost agricultural raw materials expressly for the purpose of bacterial fermentation, thereby providing a route for indirect fermentation of biomass to alcohol. There are numerous examples of raw materials which can be converted to syngas, as most types of vegetation could be used for this purpose. Suitable raw materials include, but are not limited to, perennial grasses such as switchgrass, crop residues such as corn stover, processing wastes such as sawdust, byproducts from sugar cane harvesting (bagasse) or palm oil production etc. Those of skill in the art are familiar with the generation of syngas from such starting materials. In general, syngas is generated in a gasifier from dried biomass primarily by pyrolysis, partial oxidation, and steam reforming, the primary products being CO, H₂ and CO₂. The terms “gasification” and “pyrolysis” refer to similar processes; both processes limit the amount of oxygen to which the biomass is exposed. The term “gasification” is sometimes used to include both gasification and pyrolysis.

Combinations of sources for substrate gases fed into the fermentation process may also be utilized to alter the concentration of components in the feed stream to the bioreactor. For example, the primary source of CO, CO₂ and H₂ may be syngas, which typically exhibits a concentration ratio of 37% CO, 35% H₂, and 18% CO₂, but the syngas may be supplemented with gas from other sources to enrich the level of CO (i.e., steel mill waste gas is enriched in CO) or H₂.

The symbiotic co-cultures of the present invention must be cultured under anaerobic conditions. As used herein, “anaerobic conditions” means the level of oxygen (O₂) is below 0.5 parts per million in the gas phase of the environment to which the microorganisms are exposed. One of skill in the art will be familiar with the standard anaerobic techniques for culturing these microorganisms (Balch and Wolfe, 1976, Appl. Environ. Microbiol. 32:781-791; Balch et al., 1979, Microbiol. Rev. 43:260-296).

Currently, no natural symbiotic pairings able to produce propanol or acid propionic from syngas have been found in natural environments. However, microorganisms from natural environments, when paired together under the correct nutrient conditions and selection pressures can be forced to form these “unnatural” metabolic symbiotic pairings which will produce propanol from syngas.

Symbiotic cultures for use in the invention can be generated in several ways. One approach involves using nutrient selection pressures to form a metabolic symbiosis between at least two of the microorganisms found in an environmental sample containing a mixed anaerobic microbial community. In this method, the only carbon and electron sources available for microbial growth are either syngas and/or syngas fermentation products, such as ethanol and acetate. Under these nutrient selection pressures, microorganisms capable of growing on these nutrients will be enriched.

A variation of the process for forming symbiotic associations described above involves dilution. This process allows the very slow growing C3-producing propionogens in the sample to reach a higher cell density. Dilution of enrichment cultures can proceed with either a continuously fed anaerobic fermenter or manually through serial dilutions of enrichment samples. Both dilution techniques apply the same nutrient selection pressure of carbon and electron sources described previously.

Another method for establishing a symbiotic association capable of converting syngas to propanol involves the growing of two or more defined cultures and establishing the pairing of these separate cultures. A person skilled in the art would appreciate that there are numerous methods of pairing two or more defined cultures. For example, one method involves first growing a known C1-fixing homoacetogen in a fermenter with syngas as the only carbon and electron source. In a preferred embodiment, the homoacetogen will produce ethanol and, at the same time, a known C3-producing propionogen culture is grown in a separate fermentor. Once the homoacetogen has reached steady state with respect to ethanol and/or acetate productivity, a known C3-producing propionogen culture is seeded into the fermenter.

Another method of pairing involves first growing the C3-producing propionogen in a fermenter until maximum productivity target of propionic acid+propanol has been achieved. This stage of fermentation should have syngas as the sparging gas to acclimate the culture to syngas and to provide CO₂ for propionic acid production. Once the maximum productivity target has been reached, a seed culture of the C1-fixing homoacetogen is added directly to the fermenter containing the C3-producing. Syngas mass transfer to the fermentation vessels is gradually increased to balance the gas consumption of the C1-fixing homoacetogen. The ethanol or acetate used to grow the C3-producing propionogen are gradually decreased to zero as the C1-fixing homoacetogen begins to provide this substrate. A modification of this last method of establishing a symbiotic culture involves first growing the C3-producing propionogen culture in a fermenter with a biofilm support material that is either stationary or floating within the reactor. US Patent Publication 20090035848, which is herein incorporated in its entirety, shows the use of floating support material in a moving bed bioreactor. An example of such material is the Mutag Biochips. This method allows the C3-producing microorganism to first establish a biofilm on the carrier material thereby increasing the cell retention time versus the hydraulic retention of the fermenter. Again, target propionic acid productivity is reached before seeding the fermenter with the C1-fixing homoacetogen.

The last method to establish a symbiotic culture capable of producing propanol from syngas involves the initial mixing together of two or more cultures, one of which is a C1-fixing homoacetogen capable of growing on syngas and producing ethanol and acetate. The other culture(s) is a C3-producing propionogen capable of converting ethanol or acetate to propionic acid. Ethanol and acetate feed can gradually be decreased to zero as the production of these substrates by the C1-fixing homoacetogens increases to balance the substrate consumption

A suitable medium composition used to grow and maintain symbiotic co-cultures includes a defined media formulation. The standard growth medium is made from stock solutions which result in the following final composition per Liter of medium. The amounts given are in grams unless stated otherwise. Minerals: NaCl, 2; NH₄Cl, 25; KCl, 2.5; KH₂PO₄, 2.5; MgSO₄.7H₂O, 0.5; CaCl₂.2H₂O, 0.1. Trace metals: MnSO₄.H₂O, 0.01; Fe(NH₄)₂(SO₄)₂.6H₂O, 0.008; CoCl₂.6H₂O, 0.002; ZnSO₄.7H₂O, 0.01; NiCl₂.6H₂O, 0.002; Na₂MoO₄.2H₂O, 0.0002, Na₂SeO₄, 0.001, Na₂WO₄, 0.002. Vitamins (amount, mg): Pyridoxine HCl, 0.10; thiamine HCl, 0.05, riboflavin, 0.05; calcium pantothenate, 0.05; thioctic acid, 0.05; p-aminobenzoic acid, 0.05; nicotinic acid, 0.05; vitamin B12, 0.05; mercaptoethanesulfonic acid, 0.05; biotin, 0.02; folic acid, 0.02. A reducing agent mixture is added to the medium at a final concentration (g/L) of cysteine (free base), 0.1; Na₂S.2H₂O, 0.1. Medium compositions can also be provided by yeast extract or corn steep liquor or supplemented with such liquids.

A MSBR is most commonly used in the inventive methods to enhance a close association between the C1 homoacetogen and C3 propionogen. A MSBR allows for interspecies transfer of electrons, ions and metabolites by eliminating the diffusional barrier which leads to good energetics and kinetics.

In one embodiment, the MSBR is of the Biopore type where the C1 homoacetogen and C3 propionogen are seeded inside the Biopore. The MSBR can be seeded with the mixed symbiotic culture in different sequences. Simultaneous seeding mixes the C1 homoacetogen and C3 propionogen cultures together and then seeds the membrane in one event. The microorganisms distribute relatively homogeneously within the Biopore volumes in whatever ratio they are supplied. Alternatively, the C1 homoacetogen and C3 propionogen populations can be seeded sequentially, which separates and seeds the membrane in two separate but sequential seeding events.

As illustrated in FIG. 4, a membrane arrangement 10 having biopores 11 (that ollectively make up the biopore volume) is seeded with a first microorganism 12 (black circles) and a second microorganism 13 (open circles) with the circles representing either the C3 propionogen or the C1 homoacetogen such that either microorganism can be the first culture to seed into the Biopore volumes. FIG. 4 is arranged such that the right side 14 of the membrane arrangement 10 shows simultaneous seeding while the left side 15 shows sequential seeding. FIG. 4 also show the passage of liquid up through the lumen 16 of to supply media to the microorganisms 12, 13 and the recovery of propanol therefrom. At the same time the gaseous substrate passes downwardly on the outside of the membrane 17 (shell side) to supply the needed carbon and electrons. Although gas flow on the shell side and the liquid flow though the lumen are depicted in specific directions, the gas flow and liquid flow may be in any relative direction of countercurrent flow, parallel flow or cross flow. The bioreactor system and the seeding methods used herein are further described in U.S. Patent Publication 20090215163, which is incorporated herein in its entirety.

The methods of this invention provide for a symbiotic relationship that overcomes operating pH values and the microorganism's common use of hydrogen. The symbiotic C3 microorganisms, if grown in a separate bio reactor will typically operate at a pH range of about 6 to 7. Under these conditions the C3 microorganisms produce a salt of the acid rather than the free acid. When converted to the alcohol, in a separate solventogenic bioreactor, this salt will be converted to its corresponding alkali and will raise the pH, likely beyond the optimal operating range (4.5 to 6.5) of the solventogenic bio reactor. The use of a symbiotic co-culture controls the pH within the optimal pH operating range. This control occurs because organisms in a symbiotic co-culture are in very close proximity to each other, enabling interspecies transfer of protons, electrons or other molecules across very short diffusion paths which are sometimes less than 1 micron. Hydrogen is also used by both the C1 homoacetogen and C3 propionogen—the homoacetogens for acetate and alcohol production and the propinogens for the conversion of acetate to propionate, which is thermodynamically favored and provides the primary growth energy for the syntrophs as shown in the formula below:

Acetate+HCO₃-+H++3H₂->Propionate+3H₂OdG=−18.3 Kcal

The methods of the present invention are performed in a membrane reactor. The culturing of the C1-fixing homoacetogen and C3-producing propionogen can be performed in any of several types of fermentation apparatuses that are known to those of skill in the art, with or without additional modifications, or in other styles of fermentation equipment that are currently under development. Examples include but are not limited to bubble column reactors, two stage bioreactors, trickle bed reactors, membrane reactors, packed bed reactors containing immobilized cells, etc. These apparatuses will be used to develop and maintain the C1-fixing homoacetogen and C3-producing propionogen cultures used to establish the symbiotic metabolic association in the membrane reactor. The chief requirements of such an apparatus include:
a. Axenicity;
b. Anaerobic conditions;
c. Suitable conditions for maintenance of temperature, pressure, and pH;
d. Sufficient quantities of substrates are supplied to the culture;
e. Optimum mass transfer performance to supply the gases to the fermentation medium
f. The end products of the fermentation can be readily recovered from the bacterial broth.

The fermentation reactor for culturing the microorganism for inoculation in the membrane reactor may be, for example, a traditional stirred tank reactor, a column fermenter with immobilized or suspended cells, a continuous flow type reactor, a high pressure reactor, a suspended cell reactor with cell recycle, and other examples previously listed.

The membrane bioreactors that contain the symbiotic co-culture may be arranged in a series and/or parallel reactor system. For example, multiple reactors can be useful for growing cells under one set of conditions and generating n-propanol (or other products) with minimal growth under another set of conditions. Membranes and membrane reactor arrangements are well known and examples of these apparatus are given in U.S. Pat. No. 8,058,058; U.S. Ser. No. 12/036,007 allowed Oct. 2, 2012; U.S. Pat. No. 8,017,384; U.S. Pat. No. 8,062,873; U.S. Pat. No. 8,101,387 and U.S. Pat. No. 8,309,348.

When inoculating the membrane bioreactor with a co-culture, the, fermentation of the symbiotic co-culture will be allowed to proceed until a desired level of propanol is produced in the culture media. Once established in the membrane bioreactor, the level of n-propanol produced is in the range of 2 grams/liters to 75 grams/liters and most preferably in the range of 4 grams/liters to 50 grams/liters. Production may be halted when a certain rate of production is achieved, e.g. when the rate of production of a desired product has declined due to, for example, build-up of bacterial waste products, reduction in substrate availability, feedback inhibition by products, reduction in the number of viable bacteria, or for any of several other reasons known to those of skill in the art. In addition, continuous culture techniques exist which allow the continual replenishment of fresh culture medium with concurrent removal of used medium, including any liquid products therein (i.e. the chemostat mode).

The products that are produced by the microorganisms of this invention can be removed from the culture and purified by any of several methods that are known to those of skill in the art. For example, propanol can be removed by adsorption, by distillation at atmospheric pressure or under vacuum or by other membrane based separations processes such as pervaporation, vapor permeation and the like and further processed such as by chemical/catalytic dehydration to produce propylene.

FIG. 5 illustrates a specific configuration of a bio-reactor system utilizing a membrane containing microorganisms as depicted in FIG. 4. A gas supply conduit delivers a feed gas stream 30 containing syngas at a rate recorded by a flow meter 31 to a bioreactor 33. The bioreactor 33 includes a vessel 35 that surrounds the outside of the tubular membrane elements 32. Membrane elements 32 contain the bio-pores that promote growth and maintenance of C1 fixing and the C3 producing microorganisms within a bio-layer of the membrane elements.

A feed gas distribution chamber 35 receives the feed gas stream and distributes the gas on the shell side of bioreactor 33 into direct contact with the outer surface of the membrane elements 32. The feed gas exits the vessel 35 via a line 34 such that a continuous addition of feed gas is established around the outer surface of membrane elements 32. The relative locations of the feed gas lines provide a downward direction of the bulk gas flow in the bioreactor 33.

Vessel 35 also contains a line 29 for draining liquid. Liquid may accumulate at the bottom of the vessel 35 for a variety of reasons as previously described such as condensation from moisture in the gas, flushing or purging of the membrane elements or periodic cleaning operations. Alternately line 29 may provide an outlet for liquid drainage. Liquid from condensation or flushing may be withdrawn from either location and treated for return to the fermentation liquid.

Fermentation liquid enters bioreactor 33 via a conduit 38 under pressure supplied by a pump 39 and at rate recorded by a flow meter 28. A chamber 37 distributes fermentation liquid to the tubular membranes 32 via the bottom ends of the lumens. At the top end of bioreactor 33 a chamber 43 collects the fermentation liquid from the top of the lumens for withdrawal of the liquid via a conduit 44. The relative locations of chambers 37 and 43 establish upward flow of the liquid through bioreactor 33 so that there is countercurrent flow with respect to the bulk gas flow and the liquid flow.

A line 40 withdraws a net portion of the liquid from line 44 while the remainder of the liquid returns to the bioreactor 33 via a recirculation line 36 and mixing chamber 48, a line 41 and line 38. Line 40 carries the liquid to product recovery facilities that recover liquid products. Depending on the nature of the desired product, there are a number of technologies that can be used for product recovery. For example, distillation, dephlegmation, pervaporation and liquid-liquid extraction can be used for the recovery of ethanol and n-butanol, whereas electrodialysis and ion-exchange can be used for the recovery of acetate, butyrate, and other ionic products. In all cases the product recovery step removes the desirable product from stream 40, while leaving substantial amounts of water and residual nutrients in the treated stream, part of which is returned to the bioreactor system via line 42 and mixing chamber 48.

Means for temperature and pH control for the liquid can be added anywhere along the re-circulating liquid loop, which consists of lines 38, 44, 36, and 41 as well as chambers 37, 43, and 48. A line 45 provides the nutrients needed to sustain the activity of the microorganisms to the re-circulating liquid loop chamber 48. Chamber 48 provides mixing of the nutrients and the other streams.

The flow rates of Streams 38 and 44, recirculated through the membrane unit, are selected so that there is no significant liquid boundary layer that impedes mass transfer near the lumen (liquid-facing side of the membrane.) The superficial linear velocity of the liquid tangential to the membrane should be in the range of 0.01 to 20 cm/s, preferably 0.05 to 5 cm/s, and most preferably 0.2 to 1.0 cm/s.

In all the depicted arrangements the CO and H₂/CO₂ from the syngas are utilized and a gradient for their transport from the gas feed side is created due to biochemical reaction on the membrane liquid interface. Thus the very large surface areas of the membrane pores are usable for gas transfer to the microorganisms and the product is recovered from the liquid side. Furthermore, the reaction rate, gas concentration gradient and the thickness of the microorganisms can be maintained in equilibrium because the microorganisms will maintain themselves only up to the layer where the gas is available as a result of the inherently slow growth characteristics of the microorganisms that metabolize syngas components.

The membranes can be configured into typical modules as shown as an example in FIG. 4 for hollow fibers. The gas flows in the fine fibers that are bundled and potted inside a cylindrical shell or vessel through which the liquid is distributed and circulated. Very high surface areas in the range of 1000 m² to 5000 m² per m³ can be achieved in such modules.

This invention is more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The terms used in the specification generally have their ordinary meanings in the art, within the context of the invention, and in the specific context where each term is used. Some terms have been more specifically defined to provide additional guidance to the practitioner regarding the description of the invention.

### EXAMPLES

The Examples which follow are illustrative of specific embodiments of the invention, and various uses thereof. They are set forth for explanatory purposes only, and are not to be taken as limiting the invention.

### Example 1

### C¹³-Labeled Propionic Acid Conversion to Propanol

To demonstrate that homoacetogen cultures growing on syngas convert propionic acid to propanol and other fermentation byproducts, C¹³-propionic acid experiments were performed. C¹³-propionic acid was fed to homoacetogen culture, Clostridium coskatii, at a concentration of 100 mM in a serum bottle and incubated at 37° C. Samples were withdrawn from the serum bottles at 2 hrs, 24 hrs and 1 week. GC-MC was used to identify the products containing the heavy stable isotope C¹³. C¹³ products were found in the propanol peak and there was no propanol produced without the C¹³ label. In addition there were no other products formed that contained the C¹³ heavy carbon isotope or its mass fragments demonstrating that homoacetogens can reduce propionic acid to propanol and no other end products when growing on syngas.

### Example 2

### Propionic Acid to Propanol in Homoacetogen Fermenters

An ethanol producing homoacetogen fermenter was continuously fed propionic acid to investigate the rate and yield of propanol. The initial concentration of ethanol in the fermenter was 500 mmol/L before propionic acid feed was started. Concentrations of propanol reached 167 mmol/L in the fermenter at a feed rate of 200 mmol/L/hr propionic acid. Residual propionic acid in the fermenter was 27 mmol/L; therefore the conversion efficiency to propanol was 97%. The concentration of ethanol in the fermenter steadily decreased as the concentration of propanol increased. At 167 mmol/L propanol the fermenter contained 250 mmol/L of ethanol. This ratio of alcohols demonstrates an electron balance based on the gas consumption rates of syngas in the fermenter. A production rate of propanol at steady state of 0.22 g/L/hr was achieved in the fermenter. The results show both high conversion efficiency and rates of propionic acid to propanol by homoacetogenic microorganisms growing on syngas. In addition, these results also showed no impact on syngas consumption with propanol concentrations as high as 10 g/L (167 mmol/L). These results demonstrate that in a co-fermentation with the homoacetogen partner such as C. coskatii propionic acid is readily converted to propanol and the residual acetic acid is recycled and converted to propanol by this symbiotic co culture.

### Example 3

### Propionic Acid Production from Ethanol by Propionogens in Fermenters

A fermenter was started with Clostridium neopropionicum growing on ethanol as the source of electrons and bicarbonate and ethanol as the source of carbon. Ethanol concentration in the media feed was 213 mmol/L. The fermenter reached a concentration of 89 mmol/L propionic acid, 5 mmol/L of propanol, and a residual ethanol of 27 mmol/L at steady state. This represented a conversion efficiency of 76% from ethanol to propionic acid based on a theoretical conversion stoichiometry of 1.5 moles of ethanol per mole of propionic acid produced. Other reaction products included acetic acid and small amounts of butyric acid.

These experiments demonstrate the feasibility of converting ethanol to propionic acid at high yields under syngas fermentation conditions.

### Example 4

### Propionic Acid Production by Conversion of Syngas Using Homoacetogen and Propionogen in Biopores

In this experiment a known propionogen, Clostridium propionicum, was first seeded onto an MSBR membrane in a biopore MSBR, and allowed to grow for 5 days prior to seeding with a known homoacetogen, Clostridium authoethanogenum. The MSBR used a membrane module containing membranes comprising hydrophilic polysulfone hollow fibers (Spectrum Lab, Model# M7-500S-300-01N) that have a spongy pore structure exterior surface that provides pores that serve as the biopores. The fiber has ID of 0.5 mm and OD of 0.66 mm. The membrane has nominal molecular weight cutoff of 500 k. The pores have a nominal diameter of approximately 2 microns and the spongy layer that provides the pores has an average thickness of 60 to 70 microns. The whole membrane module was 3.12 cm in diameter and 20.6 cm in length, with 0.41 m2 of total membrane surface area.

The propionogen was grown on a media consisting of minerals, vitamins and a continuous feed of approximately 0.2 g/hr of Ethanol. Typical composition of the media was Minerals: NaCl, 2; NH₄C1, 25; KCl, 2.5; KH₂PO₄, 2.5; MgSO₄.7H₂0, 0.5; CaCl2.2H₂0, 0.1. Trace metals: MnSO₄.H₂0, 0.01; Fe(NH₄)₂(SO₄)₂.6H₂0, 0.008; CoCl₂.6H₂0, 0.002; ZnSO₄.7H₂0, 0.01; NiCl₂.6H₂0, 0.002; Na₂Mo0₄-2H₂0, 0.0002, Na₂Se0₄, 0.001, Na₂W04, 0.002. Vitamins (amount, mg): Pyridoxine HCl, 0.10; thiamine HCl, 0.05, riboflavin, 0.05; calcium pantothenate, 0.05; thioctic acid, 0.05; p-aminobenzoic acid, 0.05; nicotinic acid, 0.05; vitamin B12, 0.05; mercaptoethanesulfonic acid, 0.05; biotin, 0.02; folic acid, 0.02. A reducing agent mixture was added to the medium at a final concentration 0.21 g/L) of cysteine (free base). The pH of this experiment was started at 6. In addition to the defined media reagents, 0.5 g/L yeast extract was also added to supplement unknown nutritional components for the C3-producing bacteria.

The total volume of the media recirculation tank vessel was 2.1 L. Temperature was set at 33° C. on the media side (within the membrane lumen) and 34° C. on the shell side (exterior of the membrane fibers) of the MSBR membrane. During the propionogen growth phase, the gas composition on the media side of the MSBR membrane was 20 mol % CO₂ and 80% N₂. No gas was sparged through the media but the MSBR gas was vented into the headspace of the media vessel prior to analysis on the process Mass Spectrometer.

After 122 hrs of growth of the propionogen in the MSBR under 0.25 g/h of ethanol supply, the concentration (g/l) of the primary products in the fermentor were as follows: ethanol=1.27; n-propanol=0.25; acetic acid=1.53 and propionic acid=2.25.

At 122 hours 100 mL of the homoacetogen seed was added and incorporated into the biopores of the MSBR membrane through the shell side of the MSBR, and the gas composition for the shell side was switched to syngas consisting of 56 mol % H₂, 21 mol % CO, 4.8% CO₂ and balance of CH₄, while the supply of ethanol was stopped.

At 161 hrs i.e. approximately 40 hrs after seeding with the homoacetogen and continuing fermentation with the co-cultures in the MSBR, the concentration (g/l) of the primary products in the fermentor were as follows: ethanol=3.15; n-propanol=1.24; acetic acid=2.64 and propionic acid=0.49. The uptake of H2 and CO continually increased after the seeding with the homoacetogen and at 161 hrs it had reached H2 uptake=22 mmole/l/hr and CO=12 mmole/l/hr.

Even after discontinuation of the exogenous ethanol feed, the MSBR fermentation continued to produce n-propanol at steady state levels of approximately 0.85 g/l and propionic acid at approximately 0.5 g/l for an additional 40 hrs of fermentation at which time the experiment was terminated.

This data clearly shows that the co culture of the propionogen and the homoacetogen in the MSBR enables the conversion of H2 and CO from syngas to C3 products

### Example 5

### Propanol Production by Co-Culture of a Homoacetogen and a Propionogen that Uses the Acrylate Pathway

A homoacetogenic bacterial culture of C. coskatii, grown on syngas in a fermenter and producing ethanol and acetate was mixed in with an anaerobic batch (bottle) culture of C. neopropionicum, which has the lactate acrylate pathway, grown on ethanol and producing propionate and low levels of propanol. The co-cultures, in bottles, were incubated under syngas with pH adjustment by addition of a dilute sodium bicarbonate (NaHCO₃) solution. The initial ethanol concentration in the co-cultures was approximately 180 mM (8.3 g/L), which was derived from the syngas fermentation. The initial propionate concentration was ˜3 mM (0.22 g/L), which was introduced into the co-culture mixture with the C. neopropionicum culture medium. The co-cultures were grown under syngas atmosphere of initial composition of ˜38% CO, ˜38% H₂, ˜15% CO₂ and ˜9% CH₄. The pH was adjusted periodically to maintain the level at or above pH 6.0. After 48 hrs samples were taken and analyzed. The analysis showed that ethanol was consumed and propanol production peaked at 36 mM (2.2 g/L), a level 12 times the initial molar propionate concentration, demonstrating that the propanol was derived from the syngas-produced ethanol and was not just the product of conversion of the initial propionate present. The propionate concentration also increased under these conditions to 33 mM (2.4 g/L) at day-three of incubation (when the experiment was terminated). These results indicate that a co-culture of a solventogenic syngas-metabolizing homoacetogen and an ethanol-metabolizing propionate-producing anaerobic bacterium can produce propanol from syngas-derived ethanol at a significant yield.

### Example 6

### Propanol Production by Co-Culture of a Homoacetogen and a Propioneogen that Uses the Methylmalonyl Succinate Pathway

A homoacetogenic bacterial culture of C. coskatii, grown on syngas in a fermenter and producing ethanol was mixed with an anaerobic, batch (bottle), culture of Pelobacter propionicus, which uses the methylmalonyl-succinate pathway, grown on ethanol and producing propionate and low levels of propanol. The initial ethanol concentration in the co-culture was approximately 120 mM (5.6 g/L), the majority of which was derived from the syngas fermentation. The initial propionate concentration was ˜1.8 mM, which was introduced into the co-culture mixture with the P. propionicus culture medium. The co-culture was incubated in a bottle at 30° C. with agitation under a syngas atmosphere with an initial composition of approximately 38% CO, 38% H₂, 15% CO₂ and 9% CH₄. The initial pH of the co-culture mixture was adjusted to ˜7.0 by addition of a dilute sodium bicarbonate (NaHCO₃) solution. Samples taken for analysis at the end of an 8 day incubation period showed ethanol utilization and propanol production. Approximately 40% of the original ethanol present in the mixture was consumed (47.44 mM) which resulted in a final total C3 compound (propanol+propionate) concentration of 17.5 mM. Propanol represented the majority of the C3 production with a final concentration of 14.43 mM while the propionate concentration was 3.07 mM. These concentrations represent a 13 and 1.67 times increase above initial values for propanol and propionate, respectively and a net production of 14.56 mM C3 compounds. There was no net production of C3 compounds in a control experiment where the Pelobacter propionicus cells were not present. These results demonstrate that a co-culture of a solventogenic syngas-metabolizing homoacetogen and an ethanol-metabolizing propionate-producing anaerobic bacterium can produce propanol from syngas-derived ethanol at a significant yield.

1. A method for producing propanol and/or propionate comprising exposing gaseous substrates selected from the group consisting of carbon monoxide, carbon dioxide and hydrogen or combinations thereof to a symbiotic co-culture comprising a C1-fixing microorganism and a C3-producing microorganism both contained within biopores that open to a gas contact surface located on one side of an asymmetric hydrophilic membrane and circulating a fermentation liquid on a liquid contact surface located on the side of the membrane opposite to the gas contact surface to supply nutrients to the biopores and to recover propanol from the biopores while maintaining the symbiotic co-culture at conditions effective to convert the gaseous substrate into propanol and/or propionate.

2. The method of claim 1 wherein a mixture of the C1-fixing microorganism and the C3-producing microorganism are introduced at the same time into the biopores of the asymmetric membrane.

3. The method of claim 1 wherein the C1-fixing microorganism and the C3-producing microorganism are sequentially seeded into the biopores by first introducing a culture of the C3-producing microorganism into the biopores and subsequently introducing a culture of the C1-fixing microorganism into the biopores.

4. The method of claim 1 wherein the C1-fixing microorganism and the C3-producing microorganism are sequentially seeded into the biopores by first introducing a culture of the C1-fixing microorganism into the biopores and subsequently introducing a culture of the C3-producing microorganism into the biopores.

5. The method of claim 1 wherein the C1-fixing microorganism is a solventogenic homoacetogen, using the acetyl-CoA pathway.

6. The method of claim 1 wherein the C1-fixing microorganism is selected from the group consisting of Clostridium Coskatii, Clostridium ljungdahlii, Clostridium authoethanogenium, Clostridium ragsdalei, Alkalibaculum bacchi, Clostridium thermoaceticum, and Clostridium aceticum.

7. The method of claim 1 wherein the C3-producing microorganism is a propionogen.

8. The method of claim 7 wherein the propionogen uses the lactate-acrylate pathway for the production of propionate.

9. The method of claim 7 wherein the propionogen uses the methylmalonyl-succinate pathway for the production of propionate.

10. The method of claim 1 wherein the C3-producing microorganism is selected from the group consisting of Clostridium neopropionicum, Clostridium propionicum, Pelobacter propinoicus, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosymbioticum, Pelotomaculum thermopropionicum, and Pelotomaculum schinkii.

11. The method of claim 1 wherein the gaseous substrate is syngas.
